(19) United States
(12) Patent Application Publication
(10) Pub. No.: US 2004/0241104 A1
(43) Pub. Date: Dec. 2, 2004

(54) Aerosol device comprising a cosmetic composition comprising at least one polyurethane and at least one propellant comprising dimethyl ether and at least one C3-C5 hydrocarbon

(75) Inventors: Francois Le Bourhis, Aubervilliers (FR); Francoise Pataut, Paris (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(73) Assignee: L'OREAL

(21) Appl. No.: 10/796,016

(22) Filed: Mar. 10, 2004

Related U.S. Application Data
(60) Provisional application No. 60/477,362, filed on Jun. 11, 2003.

(30) Foreign Application Priority Data
Mar. 11, 2003 (FR) 03 03000

(51) Int. Cl. A61K 9/00

(52) U.S. Cl. 424/47

(57) ABSTRACT

An aerosol device comprising:

(A) a cosmetic composition comprising, in a cosmetically acceptable medium comprising water and at least one organic solvent, at least one polyurethane, and

(B) at least one propellant comprising dimethyl ether and at least one C₃-C₅ hydrocarbon.

A process for shaping or holding a hairstyle using the aerosol device, a process for applying a lacquer to hair using the aerosol device, and a hair lacquer are also disclosed.

This application claims benefit of U.S. Provisional Application No. 60/477,362, filed Jun. 11, 2003.

Disclosed herein is an aerosol device comprising (A) a cosmetic composition comprising, in a cosmetically acceptable medium comprising water and at least one organic solvent, at least one polyurethane and (B) at least one propellant comprising dimethyl ether and at least one C₃-C₅ hydrocarbon. Further disclosed herein is a process for shaping and/or holding a hairstyle in which these devices are used, and also the use of the disclosed devices to apply a lacquer to the hair.

The cosmetic compositions most widely available on the cosmetics market for shaping and/or holding the hairstyle are spray compositions comprising a solution, usually an alcoholic solution, and at least one component, chosen, for example, from polymer resins, wherein the resins may be known as fixing components, the function of which is to form welds between the hairs, in the form of a mixture with various cosmetic adjuvants. This solution may be packaged either in a suitable aerosol container pressurized using a propellant or in a pump-dispenser bottle.

Numerous aerosol systems for fixing the hair are known. These systems may comprise a liquid phase (or fluid) and a propellant. The propellant provides the pressure that allows the liquid phase to be sprayed and to be applied to the hair in the form of a cloud of dispersed droplets.

Certain non-aqueous formulations may be characterized by the generation of a spray that is termed “gentle.” This term covers a set of sensory characteristics associated, inter alia, with the force of the spray and a reduced acoustic signature. These characteristics may be obtained in various ways applied individually or otherwise, such as reducing the pressure of the formulation or slowing the flow rate of the formulation.

To reduce to a value of 80% v/v the amount of volatile organic compounds (VOCs), such as alcohols and fluorohydrocarbons, present in spray compositions of this type, from 15% to 20% v/v of water may be introduced into these formulations. For reasons of solubility of the medium, the presence of this water in the formulation may require the use of dimethyl ether as a propellant, which may have the consequence of increasing the internal pressure, the flow rate, the force of the spray and the noise of the spray. As a result, the spray obtained can no longer be termed “gentle”.

Thus, a problem with obtaining formulations that can both produce a gentle spray and comprise dimethyl ether as a propellent gas arises. The solutions used to obtain a gentle spray with this type of formulation, which may, for example, reduce the flow rate, do not give a fully satisfactory result. The expected working qualities may be degraded: for example, the level of fixing obtained may become too weak. The introduction of hydrocarbons in combination with dimethyl ether in a formulation comprising standard fixing polymers does not solve this problem either.

Patent Application No. FR-A-2 782 635 describes aerosol devices comprising dimethyl ether as a propellant and a polycondensate comprising at least one polyurethane and/or polyurea unit as a fixing polymer. However, these formulations may not allow a gentle spray to be generated.

Similarly, U.S. Pat. No. 5,626,840 describes a hair-fixing composition comprising a linear carboxylated polyurethane, a base, a solvent and also dimethyl ether or a hydrocarbon as a propellant. This formulation may also not allow a gentle spray to be generated.

The present inventors have found, surprisingly and advantageously, that the use of (A) a cosmetic composition comprising, in a cosmetically acceptable medium comprising water and at least one organic solvent, at least one polyurethane, and (B) at least one propellant comprising dimethyl ether and at least one C₃-C₅ hydrocarbon may allow the production of a gentle spray while at the same time providing at least one of the following properties: maintaining a good level of fixing and providing good cosmetic properties such as softness or disentangling.

Disclosed herein is thus an aerosol device comprising: (A) cosmetic composition comprising, in a cosmetically acceptable medium comprising water and at least one organic solvent, at least one polyurethane, and (B) at least one propellant comprising dimethyl ether and at least one C₃-C₅ hydrocarbon.

Further disclosed herein is a process for shaping and/or holding a hairstyle, comprising using this aerosol device.

Even further disclosed herein is the use of the device to apply a lacquer to the hair.

Other subjects, characteristics, aspects and advantages of the various embodiments disclosed herein will emerge even more clearly on reading the description and the various examples that follow.

Disclosed herein is an aerosol device comprising (A) a cosmetic composition comprising, in a cosmetically acceptable medium comprising water and at least one organic solvent, at least one polyurethane, and (B) at least one propellant comprising dimethyl ether and at least one C₃-C₅ hydrocarbon.

The at least one C₃-C₅ hydrocarbon disclosed herein may, for example, be chosen from n-butane, isobutane and propane. In one embodiment, n-butane may be used.

The at least one polyurethane disclosed herein is a fixing polyurethane. The term “fixing polyurethane” means a polyurethane whose function is to impart or maintain a given shape to the hairstyle.

The at least one polyurethane may, for example, comprise at least one base repeating unit corresponding to the general formula (I) below:

—X′—B—X′—CO—NH—R—NH—CO— (I)

wherein:

X′, which may be identical or different, is chosen from O and NH;

B is chosen from divalent, substituted and unsubstituted, hydrocarbon-based radicals; and

R is a divalent radical chosen from branched and unbranched alkylene radicals chosen from C₆-C₂₀ aromatic; C₁-C₂₀ aliphatic, for example, C₁-C₆ aliphatic; and C₁-C₂₀ cycloaliphatic, for example, C₁-C₆ cycloaliphatic, alkylene radicals, wherein the alkylene radicals are optionally substituted with at least one group chosen from halogens, C₁-C₄ alkoxy groups, and C₆-C₃₀ aryl groups, for example, a phenyl group.

For example, the radical B may be chosen from C₁-C₃₀ hydrocarbon-based radicals, such as C₂-C₁₀ hydrocarbon-based radicals and may bear at least one group chosen from carboxylic functional groups and sulphonic functional groups, wherein the at least one group is in free form or partially or totally neutralized with a mineral or organic base such as alkali metal and alkaline-earth metal hydroxides, aqueous ammonia, alkylamines, alkanolamines or organic amino acids. B may, for example, be a divalent radical derived from dimethylolpropionic acid.

The radical R may, for example, be chosen from radicals corresponding to the following formulae:

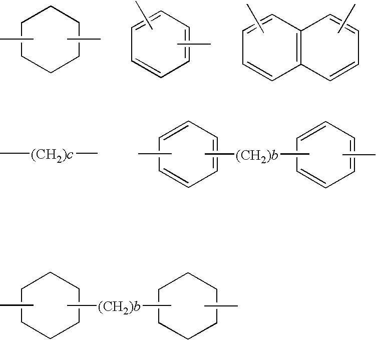

wherein b is an integer ranging from 0 to 3 and c is an integer ranging from 1 to 20, for example, from 2 to 12.

For example, R may be chosen from hexamethylene, 4,4′-biphenylenemethane, 2,4-tolylene, 2,6-tolylene, 1,5-naphthylene, p-phenylene, methylene-4,4-bis-cyclohexyl, and a divalent radical derived from isophorone.

As disclosed herein, the at least one polyurethane may comprise silicone grafts and/or silicones comprising hydrocarbon-based grafts. These various compounds may be non-ionic, cationic or amphoteric.

The at least one polyurethane disclosed herein may also, for example, comprise at least one polysiloxane block having at least one base repeating unit corresponding, for example, to the formula (II):

—X′—P—X′—CO—NH—R—NH—CO— (II)

wherein:

P is a polysiloxane segment;

X′, which may be identical or different, is chosen from O and NH; and

R is a divalent radical chosen from branched and unbranched alkylene radicals chosen from C₆-C₂₀ aromatic; C₁-C₂₀ aliphatic; for example, C₁-C₆ aliphatic; and C₁-C₂₀ cycloaliphatic, for example, C₁-C₆ cycloaliphatic, alkylene radicals, wherein the alkylene radicals are optionally substituted with at least one group chosen from halogen, C₁-C₄ alkoxy groups, and C₆-C₃₀ aryl groups, for example, a phenyl group.

The radical R may, for example, be chosen from the radicals corresponding to the following formulae:

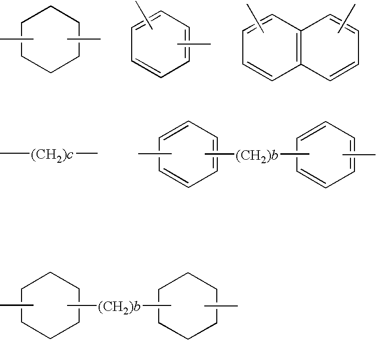

wherein b is an integer ranging from 0 to 3 and c is an integer ranging from 1 to 20, for example, from 2 to 12.

For example, the radical R may be chosen from hexamethylene, 4,4′-biphenylenemethane, 2,4-tolylene, 2,6-tolylene, 1,5-naphthylene, p-phenylene, methylene-4,4-bis-cyclohexyl, and a divalent radical derived from isophorone.

For example, the polysiloxane segment P may correspond to the general formula (VIII) below:

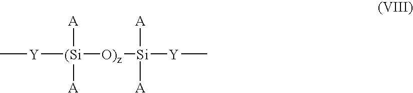

wherein:

the groups A, which may be identical or different, are chosen from C₁-C₂₀ monovalent hydrocarbon-based groups substantially free of ethylenic unsaturation, and from aromatic groups;

Y, which may be identical or different, is a divalent hydrocarbon-based group; and

z is an integer chosen such that the average molecular mass of the polysiloxane segment ranges from 300 to 10,000.

For example, the divalent group Y may be chosen from alkylene groups of formula —(CH₂)ₐ—, wherein a is an integer ranging from 1 to 10.

The groups A may be chosen from C₁-C₁₈ alkyl groups, for example, methyl, ethyl, propyl, isopropyl, butyl, pentyl, hexyl, octyl, decyl, dodecyl, and octadecyl groups; cycloalkyl groups, for example, a cyclohexyl group; aryl groups, for example, phenyl and naphthyl groups; arylalkyl groups, for example, benzyl and phenylethyl, and tolyl and xylyl groups.

The at least one polyurethane may, for example, be anionic.

Examples of the at least one polyurethane include dimethylolpropionic acid/isophorone diisocyanate/neopentyl glycol/polyesterdiols copolymer (also known as polyurethane-1, INCl name) sold under the brand name LUVISET PUR by the company BASF, and the dimethylolpropionic acid/isophorone diisocyanate/neopentyl glycol/polyesterdiols/silicone diamine copolymer (also known as polyurethane-6, INCl name) sold under the brand name LUVISET SI PUR A by the company BASF.

For example, the at least one polyurethane used may be the one sold under the name LUVISET SI-PUR ZK 553-24 by the company BASF.

The at least one polyurethane may be present in an amount ranging from 0.5% to 20% by weight, for example, from 2% to 12% by weight, relative to the total weight of all of the compositions comprised in the aerosol device.

For example, the at least one propellant may be present in an amount ranging from 20% to 70% by weight, relative to the total weight of all of the compositions comprised in the aerosol device.

The at least one C₃-C₅ hydrocarbon may be present in the at least one propellant in an amount ranging from 1 % to 30% by weight, for example, from 5% to 20% by weight, relative to the total weight of all of the compositions comprised in the aerosol device.

The dimethyl ether may be present in the at least one propellant in an amount ranging from 5% to 70% by weight, for example, from 10% to 50% by weight, relative to the total weight of all of the compositions comprised in the aerosol device.

As used here, the expression “all of the compositions comprised in the aerosol device” means the cosmetic composition A and the at least one propellant B.

The cosmetically acceptable medium is a medium comprising water and at least one organic solvent.

As used herein, the term “organic solvent” means an organic compound that is liquid at a temperature of 25° C. and at atmospheric pressure. The organic solvent may, for example, be polar.

The at least one organic solvent may, for example, be chosen from alcohols. The alcohols may, further, for example, be chosen from at least one of C₁-C₄ lower alcohols, such as ethanol, isopropanol, tert-butanol and n-butanol; and polyols, such as propylene glycol; and polyol ethers. In one embodiment, ethanol may be used.

In the cosmetically acceptable medium, water may be present in an amount ranging from 0.5% to 35% by weight, relative to the total weight of all of the compositions comprised in the aerosol device. The organic solvent proportion of the mixture ranges from 1% to 70% by weight, for example, from 15% to 65% by weight, and even further, for example, from 30% to 60% by weight, relative to the total weight of all of the compositions comprised in the aerosol device.

The cosmetic composition disclosed herein may also comprise at least one adjuvant chosen from non-polyurethane fixing polymers, such as silicones in soluble, dispersed and microdispersed forms; non-ionic, anionic, cationic and amphoteric surfactants; ceramides and pseudoceramides; vitamins and provitamins including panthenol; plant, animal, mineral and synthetic oils; waxes other than ceramides and pseudoceramides; water-soluble and liposoluble, silicone and non-silicone sunscreens; glycerol; mineral and organic, colored and uncolored pigments; permanent and temporary dyes; nacreous agents and opacifiers; sequestering agents; plasticizers; solubilizing agents; acidifying agents; basifying agents; mineral and organic thickeners; antioxidants; hydroxy acids; penetration agents; fragrances; fragrance solubilizers (peptizers); preserving agents; anticorrosion agents; and treatment agents.

A person skilled in the art will take care to select the optional additive(s) and the amount thereof such that the addition(s) does not harm the properties of the compositions disclosed herein.

The at least one adjuvant may be present in the cosmetic composition disclosed herein in an amount ranging from 0 to 20% by weight, relative to the total weight of the cosmetic composition.

The compositions disclosed herein are used as compositions for shaping and/or holding the hair.

Further disclosed herein is the use of the composition vaporized by the aerosol device disclosed herein, as a hair lacquer.

Even further disclosed herein is a hair care process for shaping and/or holding a hairstyle, comprising the use of the aerosol device described herein.

Further disclosed herein is the use of the aerosol device to apply a lacquer to the hair by vaporizing its contents.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements. The following examples are intended to illustrate the invention without limiting the scope as a result. The percentages are given on a weight basis.

### EXAMPLE 1

The present inventors prepared a device in accordance with the present disclosure, comprising formulation 1 below:

| | |
|---|---|
| LUVISET SI-PUR ZK 553-24 resin (from BASF) (expressed | 6.00 |
| as weight of polymer) | |
| PARSOL MOX (ethylhexyl methoxycinnamate) (from Roche | 0.05 |
| Vitamins) | |
| Demineralized water (contained in the polymer in its | 12 |
| commercial form) | |
| Demineralized water for formulation | 3 |
| Fragrance | 0.10 |
| n-Butane | 15 |
| Dimethyl ether | 25 |
| Absolute ethyl alcohol qs | 100 |

### EXAMPLE 2

The present inventors prepared a device in accordance with the present disclosure, comprising formulation 2:

| | |
|---|---|
| LUVISET SI-PUR ZK 553-22 resin (from BASF) (expressed | 6.00 |
| as weight of polymer) | |
| PARSOL MCX (ethylhexyl methoxycinnamate) (from Roche | 0.05 |
| Vitamins) | |
| Demineralized water (contained in the polymer in its | 12 |
| commercial form) | |
| Demineralized water for formulation | 3 |
| Fragrance | 0.10 |
| n-Butane | 15 |
| Dimethyl ether | 25 |
| Absolute ethyl alcohol qs | 100 |

Formulations 1 and 2 packaged in an aerosol device and sprayed onto hair made it possible to obtain a spray termed “gentle” and gave the hair very good hold.

What is claimed is:

1. An aerosol device comprising:
(A) a cosmetic composition comprising, in a cosmetically acceptable medium comprising water and at least one organic solvent, at least one polyurethane, and
(B) at least one propellant comprising dimethyl ether and at least one C₃-C₅ hydrocarbon.

2. The aerosol device according to claim 1, wherein the at least one C₃-C₅ hydrocarbon is chosen from at least one of n-butane, isobutane and propane.

3. The aerosol device according to claim 2, wherein the at least one C₃-C₅ hydrocarbon is n-butane.

4. The aerosol device according to claim 1, wherein the at least one polyurethane comprises at least one base repeating unit corresponding to the formula (I) below:
—X′—B—X′—CO—NH—R—NH—CO— (I)
wherein:
X′, which may be identical or different, is chosen from O and NH;
B is chosen from divalent, substituted and unsubstituted, hydrocarbon-based radicals; and
R is a divalent radical chosen from branched and unbranched alkylene radicals chosen from C₆-C₂₀ aromatic, C₁-C₂₀ aliphatic, and C₁-C₂₀ cycloaliphatic, alkylene radicals, wherein the alkylene radicals may be optionally substituted with at least one group chosen from halogen, C₁-C₄ alkoxy and C₆-C₃₀ aryl groups.

5. The aerosol device according to claim 4, wherein the C₁-C₂₀ aliphatic alkylene radicals are chosen from C₁-C₆ aliphatic alkylene radicals.

6. The aerosol device according to claim 4, wherein the C₁-C₂₀ cycloaliphatic alkylene radicals are chosen from C₁-C₆ cycloaliphatic alkylene radicals.

7. The aerosol device according to claim 4, wherein, in defining R, the at least one group is a phenyl group.

8. The aerosol device according to claim 4, wherein the radical B is chosen from divalent C₁-C₃₀ hydrocarbon-based radicals.

9. The aerosol device according to claim 4, wherein R is chosen from hexamethylene, 4,4′-biphenylenemethane, 2,4-tolyene, 2,6-tolylene, 1,5-naphthylene, p-phenylene, methylene-4,4-bis-cyclohexyl, and a divalent radical derived from isophorone.

10. The aerosol device according to claim 1, wherein the at least one polyurethane comprises at least one base repeating unit corresponding to the general formula (II):
—X′—P—X′—CO—NH—R—NH—CO— (II)
wherein:
P is a polysiloxane segment;
X′, which may be identical or different, is chosen from O and NH; and
R is a divalent radical chosen from branched and unbranched alkylene radicals chosen from C₆-C₂₀ aromatic, C₁-C₂₀ aliphatic, and C₁-C₂₀ cycloaliphatic, alkylene radicals, wherein the alkylene radicals are optionally substituted with at least one group chosen from halogen, C₁-C₄ alkoxy, and C₆-C₃₀ aryl groups.

11. The aerosol device according to claim 10, wherein the C₁-C₂₀ aliphatic alkylene radicals are chosen from C₁-C₆ aliphatic alkylene radicals.

12. The aerosol device according to claim 10, wherein the C₁-C₂₀ cycloaliphatic alkylene radicals are chosen from C₁-C₆ cycloaliphatic alkylene radicals.

13. The aerosol device according to claim 1, wherein the at least one polyurethane is anionic.

14. The aerosol device according to claim 1, wherein the at least one polyurethane is present in the cosmetic composition in an amount ranging from 0.5% to 20% by weight, relative to the total weight of all the compositions comprised in the aerosol device.

15. The aerosol device according to claim 14, wherein the at least one polyurethane is present in the cosmetic composition in an amount ranging from 2% to 12% by weight, relative to the total weight of all of the compositions comprised in the aerosol device.

16. The aerosol device according to claim 1, wherein the at least one propellant is present in the cosmetic composition in an amount ranging from 20% to 70% by weight, relative to the total weight of all of the compositions comprised in the aerosol device.

17. The aerosol device according to claim 1, wherein the at least one C₃-C₅ hydrocarbon is present in the at least one propellant in an amount ranging from 1% to 30% by weight, relative to the total weight of all the compositions comprised in the aerosol device.

18. The aerosol device according to claim 17, wherein the at least one C₃-C₅ hydrocarbon is present in the at least one propellant in an amount ranging from 5% to 20% by weight, relative to the total weight of all of the compositions comprised in the aerosol device.

19. The aerosol device according to claim 1, wherein the dimethyl ether is present in the at least one propellant in an amount ranging from 5% to 70% by weight, relative to the total weight of all the compositions comprised in the aerosol device.

20. The aerosol device according to claim 19, wherein the dimethyl ether is present in the at least one propellant in an amount ranging from 10% to 50% by weight, relative to the total weight of all of the compositions contained in the aerosol device.

21. The aerosol device according to claim 1, wherein the at least one organic solvent is chosen from alcohols.

22. The aerosol device according to claim 21, wherein the alcohols are chosen from at least one of lower C₁-C₄ alcohols, polyols and polyol ethers.

23. The aerosol device according to claim 22, wherein the C₁-C₄ alcohol is ethanol.

24. The aerosol device according to claim 1, wherein the cosmetic composition comprises from 0.5% to 35% by weight of water, relative to the total weight of all of the compositions comprised in the aerosol device.

25. The aerosol device according to claim 1, wherein the cosmetic composition comprises from 1% to 70% by weight of the at least one organic solvent, relative to the total weight of all of the compositions comprised in the aerosol device.

26. The aerosol device according to claim 25, wherein the cosmetic composition comprises from 15% to 65% by weight of the at least one organic solvent, relative to the total weight of all of the compositions comprised in the aerosol device.

27. The aerosol device according to claim 26, wherein the cosmetic composition comprises from 30% to 60% by weight of the at least one organic solvent, relative to the total weight of all of the compositions comprised in the aerosol device.

28. The aerosol device according to claim 1, wherein the cosmetic composition comprises at least one adjuvant chosen from non-polyurethane fixing polymers; silicones in soluble, dispersed and microdispersed form; nonionic, anionic, cationic and amphoteric surfactants; ceramides and pseudoceramides; vitamins and provitamins; plant, animal, mineral and synthetic oils; waxes other than ceramides and pseudoceramides; water-soluble and liposoluble, silicone and non-silicone sunscreens; glycerol; mineral and organic, colored and uncolored pigments; permanent and temporary dyes; nacreous agents and opacifiers; sequestering agents; plasticizers; solubilizing agents; acidifying agents; basifying agents; mineral and organic thickeners; antioxidants; hydroxy acids; penetration agents; fragrances; fragrance solubilizers; preserving agents; anticorrosion agents; and treatment agents.

29. The aerosol device according to claim 28, wherein the vitamins and pro-vitamins are chosen from panthenol.

30. A method for shaping and/or holding a hairstyle, comprising:
providing an aerosol device comprising,
(A) a cosmetic composition comprising, in a cosmetically acceptable medium comprising water and at least one organic solvent, at least one polyurethane, and
(B) at least one propellant comprising dimethyl ether and at least one C₃-C₅ hydrocarbon,
applying the contents of the aerosol device onto the hair by vaporizing the contents of the aerosol device onto the hair, in an amount sufficient to hold or shape a desired hairstyle.

31. A hair lacquer, wherein the hair lacquer is obtained by vaporizing the contents of an aerosol device comprising,
(A) a cosmetic composition comprising, in a cosmetically acceptable medium comprising water and at least one organic solvent, at least one polyurethane, and
(B) at least one propellant comprising dimethyl ether and at least one C₃-C₅ hydrocarbon.

32. A method for applying a lacquer to the hair comprising,
providing an aerosol device comprising,
(A) a cosmetic composition comprising, in a cosmetically acceptable medium comprising water and at least one organic solvent, at least one polyurethane and
(B) at least one propellant comprising dimethyl ether and at least one C₃-C₅ hydrocarbon,
vaporizing the contents of the aerosol device onto the hair, whereby a lacquer is formed on the hair.
